(19) United States
(12) Patent Application Publication
(10) Pub. No.: US 2011/0319467 A1
(43) Pub. Date: Dec. 29, 2011

(54) Absorption Enhancement of Statins and Omega Fatty Acids

(76) Inventor: Bhiku Patel, Chandler, AZ (US)

(21) Appl. No.: 13/165,738

(22) Filed: Jun. 21, 2011

Related U.S. Application Data
(60) Provisional application No. 61/357,649, filed on Jun. 23, 2010.

(51) Int. Cl. A61K 31/40; A61K 31/20

(52) U.S. Cl. 514/423; 514/560

(57) ABSTRACT

A composition and method to enhance absorption and bioavailability of Statin and Omega fatty acids consumed by humans and in an anhydrous base is presented.

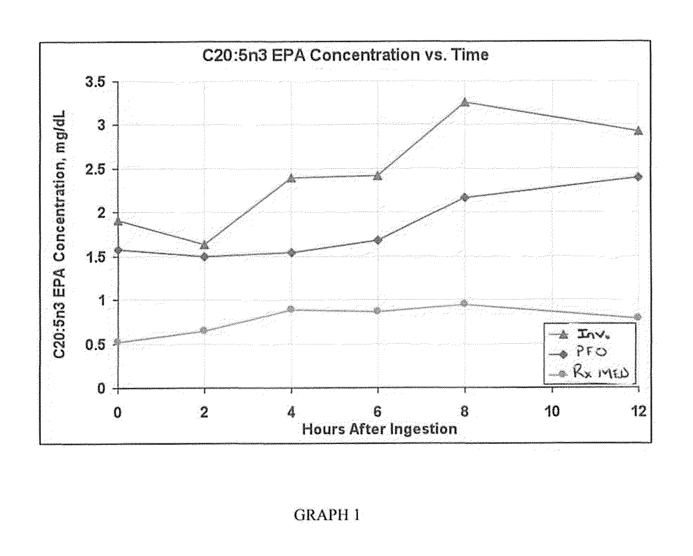

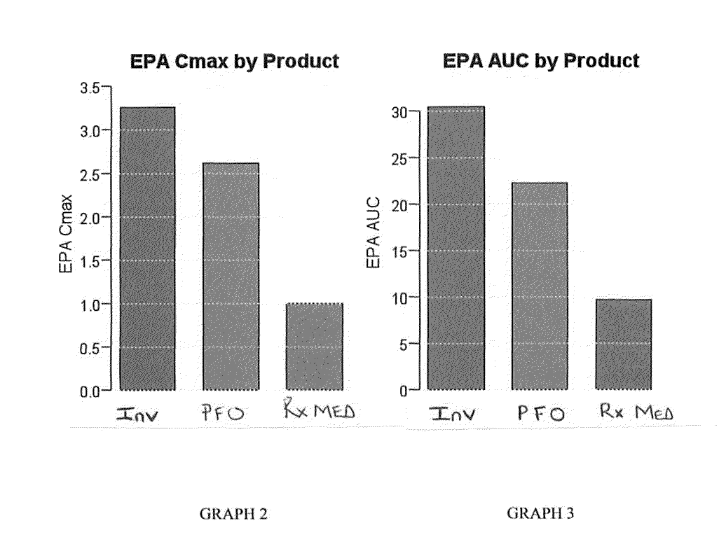

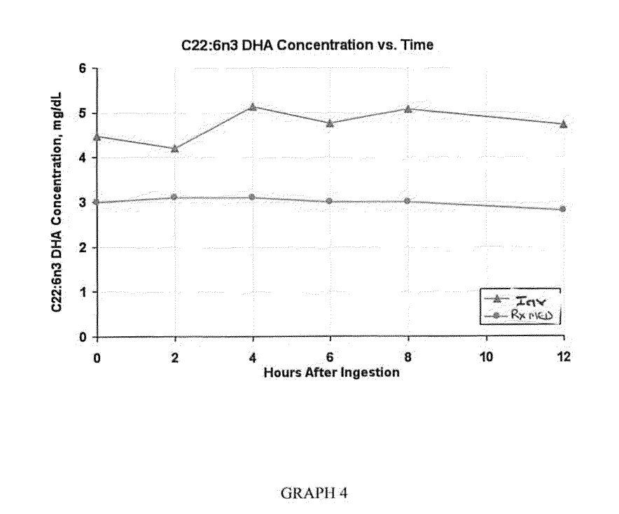

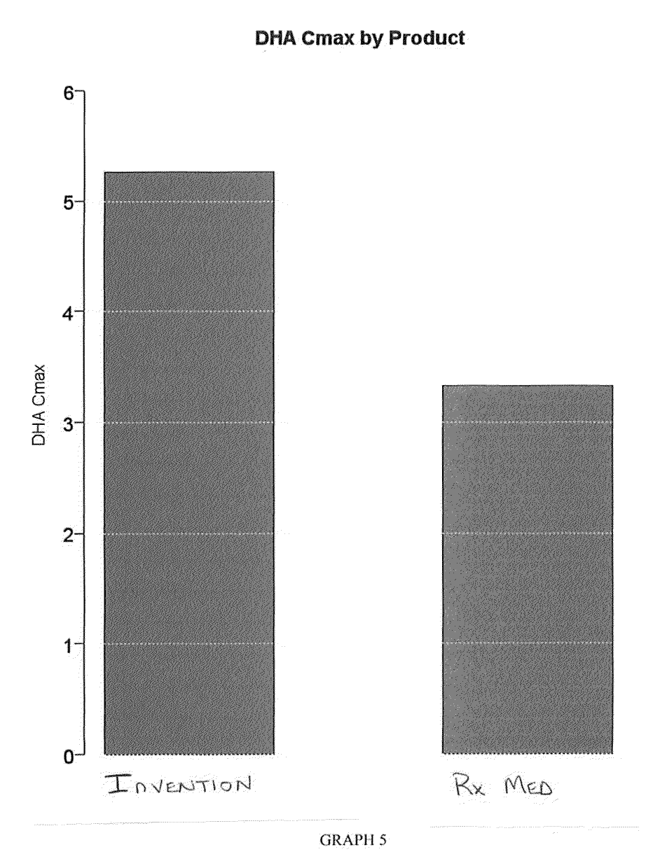

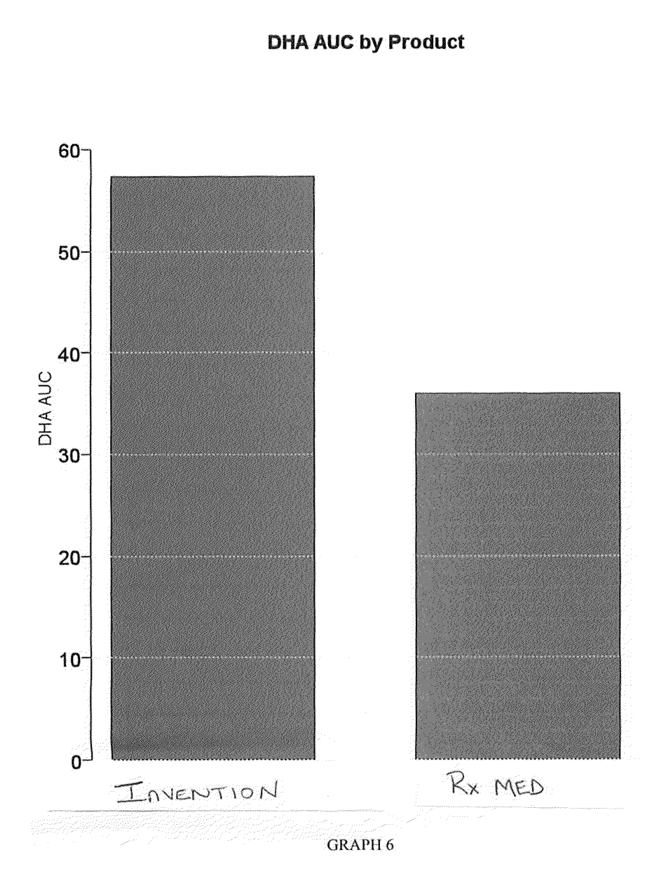

### I. TECHNICAL FIELD

The present invention relates to enhance absorption of Statin and Omega fatty acids consumed by humans and animals and, more particularly, to a composition that enhances the bioavailability of the Statin and Omega fatty acids through the mixing of the edible oil with certain types of emulsifiers in an anhydrous base.

### II. BACKGROUND OF THE INVENTION AND PRIOR ART

Statins are among the most commonly prescribed drugs in medicine. Clinical studies have shown that statins significantly reduce the risk of heart attack and death in patients with proven coronary artery disease (CAD), and can also reduce cardiac events in patients with high cholesterol levels who are at increased risk for heart disease. While best known as drugs that lower cholesterol, statins have several other beneficial effects that may also improve cardiac risk, and that may turn out to be even more important than their cholesterol-reducing properties. The statin drugs are:
atorvastatin (sold as Lipitor®, a product of Pfizer Inc.)
fluvastatin (sold as Lescol®, a product of Novartis Pharmaceuticals Corporation)
lovastatin (sold as Mevacor®, a product of Merck & Co., Inc.)
pravastatin (sold as Pravachol®, a product of the Bristol-Myers Squibb Company)
simvastatin (sold as Zocor®, a product of Merck, Sharp & Dohme Corporation)
rosuvastatin (sold as Crestor®, a product of AstraZeneca Pharmaceuticals LP)

Most people think of statins primarily as cholesterol-lowering drugs. Statins improve blood cholesterol levels primarily by inhibiting a liver enzyme called HMG Co-A reductase, thus reducing the liver's ability to make cholesterol. Statins cause a significant reduction in LDL “bad” cholesterol levels, a moderate reduction in triglyceride levels, and a small increase in levels of HDL cholesterol (“good” cholesterol).

In addition to lowering cholesterol, however, statins have several other effects that are helpful in patients known or likely to have CAD. These beneficial effects include:
Reducing the size of plaques in the arteries.
Stabilizing plaques, so they are less likely to rupture (and therefore less likely to cause acute heart attacks).
Reducing inflammation (which is now thought to be an important component of plaque formation and rupture).
Reducing CRP levels
Decreasing blood clot formation (Blood clot formation at the site of plaque rupture is the cause of most heart attacks).
Improving overall vascular function

In addition, studies have reported other possible benefits from statins, including a reduced incidence of Alzheimer's disease, particular benefits in diabetics, prevention of cataracts, and reducing blood pressure.

The most common side effects of the statins are gastrointestinal—nausea, gas, upset stomach. Less common are headache, dizziness, rash, and sleep disturbances. Statins also cause elevations in liver enzymes in about 1 in 100 patients. While blood tests should be checked after a few weeks of treatment, there is little evidence that statins ever cause serious or permanent liver damage.

Statins cause a muscle disorder producing muscle weakness, and occasionally pain, in about 1 in 1,000 patients. In the large majority of cases, the symptoms resolve if the statin is stopped, or if the dose is reduced. Rarely, sufficient muscle damage can result in kidney failure or death.

Accordingly, reducing the dosage of a statin to the lowest effective level is a primary therapeutic goal.

Another category of health oriented ingredients known to have positive cardiovascular effects are omega fatty acids. These fatty acids are most often seen in the form of edible oil. These oils, or lipids, are generally anhydrous. More recent studies also show promise in the use of these oils for controlling blood pressure, aiding in the treatment of diabetes mellitus, and assisting in bone structure maintenance. There is further evidence mounting for the use of these oils in other areas of health, including, for example, brain function, arthritis, immune system maintenance, and cancer prevention.

Omega fatty acids are essential fatty acids (“EFA”) as they are required for good nutritional balance but they are not produced by the body; they must be obtained from other sources. These essential fatty acids are found in many sources, including fish and crustacean marine sources, plants, algae, and animals.

The two main categories of omega fatty acids are omega-3 and omega-6. The Omega-3 fatty acids are alpha-linolenic acid (“ALA”), stearidonic acid, eicosapentaenoic acid (“EPA”) and docosahexaenoic acid (“DHA”). Alpha-linolenic acid is found mainly in flaxseed oil, canola oil, soybeans, walnuts, hemp seeds, and dark green leafy vegetables. Stearidonic acid is found in rarer types of seeds and nuts, including black currant seeds. EPA and DHA are present in cold-water fish, including salmon, trout, sardines, anchovies, mackerel and cod.

Omega-6 fatty acids are more common in the American diet than the Omega-3 EFAs. These include linoleic acid, which is found in safflower, olive, almond, sunflower, hemp, soybean, walnut, pumpkin, sesame, and flaxseed oils; Gamma-linolenic acid (GLA), which is found in some seeds and evening primrose oil; and, arachidonic acid (AA) which is present in meat and animal products.

Both types of EFAs, Omega-3 and Omega-6 fatty acids, are necessary in a healthy diet. However, it has been observed that deficiencies in EFAs have occurred over the years. This is more so true with the Omega-3 fatty acids for the reason that most Westerns may get an adequate amount of Omega-6 through the consumption of meat products, but do not ingest enough Omega-3 fatty acids due to lower consumption of fish, seeds, and vegetables.

This has a vicious cycle effect because not only are both types of EFA necessary for good health, Omega-6 fatty acids negatively affect the metabolism of Omega-3 fatty acid, thereby all but negating the Omega-3 fatty acids that are consumed, and an over abundance of Omega-6 fatty acids may cause an increase in prostaglandin product which, in excessive amounts, can have deleterious health effects. Experts recommend that omega-3 and omega-6 EFAs be present in the diet in a ratio of around one to three. Americans, for example, consume a ratio as high as one to 40. Thus, the need for greater amounts of omega-3 EFAs in the diet has increased.

This has resulted in a push to increase the dietary intake of Omega-3 fatty acids. While the push has been to have people eat a healthy diet, often it is either not possible or, more often, it does not happen. Accordingly, people turn to dietary or medicinal help. As a result, Omega-3 dietary supplements and, to a lesser degree, omega-3 containing medications, have become a staple in many households.

Consumer complaints regarding these supplements and medications are that they are too large and therefore uncomfortable to take and that they are unpalatable because of taste. From a scientific standpoint there is interest in making as much of the EFA bioavailable as possible so as to get the maximum effect in a short amount of time.

Attempts to address these issues have, until now, mainly revolved around purifying the oil to make it higher quality with the goal to reduce the amount of oil having to be consumed and to reduce the after-taste. From a scientific standpoint, not much research has gone into increasing the bioavailability and absorption of the oil, and, following, the EFA.

Accordingly, there is need for a composition that increases the bioavailability of EFAs and other edible oils while at the same time shortening the time for absorption, and potentially reducing the amount of oil necessary to be consumed in order to obtain a desired result in conjunction with a statin drug that is delivered simultaneously at a lower dosage than heretofore seen in the arts. Such a composition is provided for in the present invention.

### III. OBJECTS AND ADVANTAGES OF THE PRESENT INVENTION

It is an object of the present invention to provide a composition that enhances the bioavailability of omega fatty acids and statin drugs in an anhydrous or lipid environment.

It is further an object of the present invention to provide a composition that shortens the time of absorption of the fatty acid and statin drug.

It is further an object of the present invention to provide a finely dispersed oil and statin drug combination.

It is further an object of the present invention to enable the reduction in the amount of a statin drug and omega fatty acid.

The advantages offered by the present invention include but are not limited to maximizing the amount of bioavailable consumed statin drug and omega fatty acid while delivering the two components at potentially lower dosages.

### IV. SUMMARY OF THE INVENTION

The present invention comprises a composition for increasing the bioavailability of statin drugs and omega fatty acids in humans and animals comprising adding a first emulsifier and a second emulsifier in a ratio ranging from about 1:1 to about 4:1, preferably, the first emulsifier and the second emulsifier are mixed in a ratio of about 2:1, and an anhydrous base, with the final mixture of the three ingredients being in a ratio ranging from about 99:1 to about 9:1. Preferably, the ratio of the two emulsifiers together and the edible oil is about 12.333:1. Preferably, the first emulsifier is polyoxyethylene sorbitan monooleate and the second emulsifier is tocopheryl polyethylene glycol succinate. The anhydrous base can be of any type, with currently known sources being animal oils, vegetable oils, marine-based oils, and algae oils. The anhydrous bases can be used either singly or in combination with one another. To the emulsifier/anhydrous base mixture above is added a therapeutically effective amount of a statin drug. The statin drug can be either in suspension or solubilized polyethoxylated castor oil, polyethylene glycols, propylene glycol, fatty acids and esters, ethoxylated fatty acids and esters, alcohols, and their derivatives. (throughout reference to oil synonymous with anhydrous base and lipid)

Through the use of the inventive composition, a corollary to increasing the bioavailability of the fatty acid-statin drug combination is that it reaches the blood stream more quickly than heretofore seen as well as potentially decreasing the amount of both omega fatty acids and statin drug required.

There has been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention.

### V. BRIEF DESCRIPTION OF THE GRAPHS

Graph 1 illustrates eicosapentaenoic acid (“EPA”) Concentration versus Time.

Graph 2 illustrates EPA Maximum Concentration (“Cₘₐₓ”).

Graph 3 illustrates EPA Area Under the Curve (“AUC”).

Graph 4 illustrates docosahexaenoic acid (“DHA”) Concentration versus Time.

Graph 5 illustrates DHA Cₘₐₓ.

Graph 6 illustrates DHA AUC.

### VI. DETAILED DESCRIPTION OF THE PREFERRED EMBODIMENT

Before explaining the preferred embodiment of the present invention in detail, it is to be understood that the present invention is not limited in its application to the details of formulations and arrangements of the components set forth in the following description. The present invention is capable of other embodiments and of being practiced and carried out in various ways. Also, it is to be understood that the phraseology and terminology employed herein are for the purpose of description and should not be regarded as limiting. It is also to be understood that where ranges are provided for the various agents and drug examples, they are approximate ranges and are not to be limiting except where noted otherwise.

The present invention addresses the problem of omega fatty acid and statin drug bioavailability. To overcome limitations in absorption and bioavailability of these constituients, the Inventor has discovered that creating a finely dispersed micro-emulsion in turn creates an increase in oil surface area per volume, thereby enabling greater gastrointestinal uptake of the oil/stain drug combination. Through the use of emulsifiers that do not create a permanent binding bioavailability is increased. The present invention is in an anhydrous environment wherein an anhydrous base or lipid is utilized.

The anhydrous base is preferable an Oil having omega fatty acid components, and as contemplated by the invention include oils from the vast variety of sources, including without limitation, marine (such as fish and crustacean), animal, plant, and algae. Principally, the oils are selected for their Omega fatty acid content, and as is the current trend, Omega-3 fatty acids. Among the Omega-3 fatty acids in current demand are eicosapentaenoic acid (“EPA”) and docosahexaenoic acid (“DHA”). While Omega-3 fatty acids and EPA and DHA are used throughout, this is in no way meant to limit the scope and spirit of the invention. As those skilled in the arts will quickly understand, the principles taught herein will apply to any edible oil, whether it contains Omega-3 fatty acid or not. For non-limiting example, other essential fatty acids include Omega-6 fatty acids, will also work well within the principles of the Invention.

The statin drugs, also known as HMG-CoA reductase inhibitors because they interfere with HMG-CoA reductase, an enzyme necessary for the body's manufacture of cholesterol, contemplated by the Inventors include but are not limited to: atorvastatin, fluvastatin, lovastatin, pravastatin, simvastatin, and rosuvastatin.

To create a micro-emulsion adequate to provide the necessary results, the Inventor has discovered that two emulsifiers are required. A first emulsifier should be a nonionic surfactant and emulsifier. There is a wide range of such emulsifiers from which to choose. Key to the selection is that the first emulsifier be suitable for human or animal ingestion (throughout, the terms consumption and ingestion are used interchangeable and mean to take orally.) The Inventor has discovered that the preferred first emulsifier is polyoxyethylene sorbitan monooleate. The first emulsifier produces dispersion.

A second emulsifier is also required. The Inventor has discovered that employing esterfied Vitamin E works well. While esterfied Vitamin E in its various forms may be employed in the invention, the Inventor has discovered that the preferred form is tocopheryl polyethylene glycol succinate. The second emulsifier enables increased uptake.

Both of the preferred emulsifiers are well known in the industry. Polyoxyethylene sorbitan monooleate has been used for many years in the food and pharmaceutical industries. It is most commonly sold under the trade name polysorbate 80 and is widely available. It is approved by the U.S. Food and Drug Administration as an inactive ingredient and is well tolerated in oral compositions. Tocopheryl polyethylene glycol succinate is sold by several companies but was first developed by the Eastman Company and sold under the trademark ‘Vitamin E TPGS NF.’ It was developed as a water soluble emulsifier to aid in the absorption of lipid-based drugs, such as cyclosporin. Since its invention, it has been used in many products.

However, until the present invention the combination use of polyoxyethylene sorbitan monooleate and tocopheryl polyethylene glycol succinate to enhance the bioavailability of edible oils has not been taught.

Individually, each of these emulsifiers will cause at least some dispersion but it is the heretofore unknown mixture of the two emulsifiers and oil that creates the micro-emulsion necessary to increase uptake and, following, the bioavailability of the oil. Using the Inventive Composition, a fine micro-emulsion is created that enables almost complete dispersion in water, a result unseen with either of the two emulsion separately or known in the prior art. The result is gained by the emulsion properties of the tocopheryl polyethylene glycol succinate to create a fine dispersion and the polyoxyethylene sorbitan monooleate to reduce further the interfacial tension that then enables an oil-in-water micro-emulsion of a level not heretofore seen in the art.

When considering the selection of emulsifiers to use in combination to create a suitable micro-emulsion, the Inventor discovered that surface activity of the emulsifiers on the oil was an important element. Increasing the surface area of the oil enabled increased bioavailability but only to a point. Once surface area exceeds a certain value, no additional benefit is gained in absorption and, therefore, bioavailability. In fact, bioavailability can be decreased.

To determine the suitable range of surface area activity, the Inventor employed hydrophilic lipophilic balance (HLB) values. HLB is a widely accepted method for providing a measure of the surface activity of organic molecules. HLB values for emulsifiers range from about 2 to about 40. The Inventor discovered that emulsifiers in the range of about 10 to about 30 are suitable for use with the invention, with a range of about 12 to about 16 providing the best results.

Polyoxyethylene sorbitan monooleate has an HLB value of about 15 and tocopheryl polyethylene glycol succinate has an HLB value about 13. Thus, the average HLB value for the combination of the two emulsifiers is about 14 when they are found in a 1:1 polyethylene glycol succinate: tocopheryl polyethylene glycol succinate ratio and about 14.6 when they are in a 4:1 polyethylene glycol succinate: tocopheryl polyethylene glycol succinate ratio. Accordingly, the preferred HLB range is from about 14 to about 14.6.

To demonstrate the effectiveness of the combination of these two emulsifiers and edible oils, the Inventor developed an experiment to illustrate the increased bioavailability of edible oil, using EPA and DHA as markers. In the experiment, subjects were cleared of blood stream detectable levels of EPA and DHA. The Human subjects were then randomly given either the Inventive composition, a purified fish oil, or a fish oil-based prescription medication under physician supervision. Blood level readings for both EPA and DHA were then taken at 2, 4, 6, 8, 10, and 12 hours.

EPA levels for the inventive composition were significantly above those for both the purified fish oil and the prescription medication at every reading. The following table 1 illustrates the rate of blood level concentration (mg/dl/hr) for each of the three test materials:

TABLE 1

| Inventive Composition | Purified Fish oil | Prescription Medication |
|---|---|---|
| 0.253 | 0.10 | 0.06 |

The maximum blood concentration (Cₘₐₓ) for the Inventive Composition EPA is also greater than that of the purified fish oil and the prescription medication. The following table 2 shows the Cₘₐₓ (mg/dl) for each of the three test materials:

TABLE 2

| Inventive Composition | Purified Fish oil | Prescription Medication |
|---|---|---|
| 3.27 | 2.62 | 1.00 |

The Area Under Curve (AUC) for the three test materials and, again demonstrates the superiority of the Inventive Composition. Table 3 shows the average AUC₀₋₁₂ (mg/dl/hr) for the three test materials:

TABLE 3

| Inventive Composition | Purified Fish oil | Prescription Medication |
|---|---|---|
| 30.40 | 22.30 | 9.70 |

DHA readings were then taken to further demonstrate the Inventive Composition effectiveness. DHA levels for the inventive composition were significantly above those of the prescription medication at every reading.

Maximum blood concentration (Cₘₐₓ) for the Inventive Composition DHA is also greater than that of the prescription medication. The following table 4 shows the Cₘₐₓ (mg/dl) for the two test materials:

TABLE 4

| Inventive Composition | Prescription Medication |
|---|---|
| 5.26 | 3.34 |

The Area Under Curve (AUC) for the two test materials and, again demonstrates the superiority of the Inventive Composition. The AUC₀₋₁₂ for the Inventive Composition is 57.3 mg/dl/hr. and for the prescription medication is 33.0 mg/dl/hr.

In mixing of the two emulsifiers with oil to create greater bioavailability, the ratios of the two emulsifiers and oil in creating an end product with maximum effectiveness at a minimum use of emulsifiers have been considered. The Inventor has discovered that an emulsifier ratio in the range of polyoxyethylene sorbitan monooleate:tocopheryl polyethylene glycol succinate of about 1:1 to about 4:1 is useful. The preferred ratio is 2:1.

In mixing the three components together, the emulsifier combination, which can be done as a separate step and added to the oil or individually added to the oil, the Inventor has discovered that the final mixture of oil:emulsifiers should be about 99:1 to about 9:1. Levels above 99:1 do not allow adequate dispersion to take place and level below 9:1 cause gelling of the oil-emulsifier combination. The preferred ratio is about 12.333:1.

Once the emulsion is complete, a selected statin drug is introduced into the emulsion, either in suspension or by solubilization, depending on the base used and the amount and type of statin drug.

Once the fatty acid/statin drug compenation is complete, the statin drug is made more bioavailable since its uptake is increased as the oil is absorbed in the gastrointestinal tract. Accordingly, the amount of statin drug required can be reduced, which in turn, reduces the risk of a potentially devastating side effect and, beneficially reduces compliance issues as a result of the fatty acid and statin drug being delivered in a single gelcap.

It is to be understood, however, that even though numerous characteristics and advantages of the preferred and alternative embodiments have been set forth in the foregoing description, together with details of the structure and function of the embodiments, the disclosure is illustrative only, and changes may be made in detail within the principles of the invention to the full extent indicated by the broad general meaning of the terms in which the appended claims are expressed.

We claim:

1. A composition for increasing the bioavailability of statin drugs and omega fatty acids in humans and animals comprising a microemulsion further comprising a first emulsifier, a second emulsifier, and an anhydrous base.

2. The composition of claim 1 wherein the anhydrous base is a lipid.

3. The drug of claim 2 wherein the statin drug is atarvostatin.

4. The composition of claim 1 wherein the first emulsifier and the second emulsifier are in a ratio ranging from about 1:1 to about 4:1.

5. The composition of claim 1 wherein the first emulsifier and the second emulsifier are in a ratio of about 2:1.

6. The composition of claim 1 wherein the first emulsifier and the second emulsifier, in combination, and anhydrous base are in a ratio ranging from about 99:1 to about 9:1.

7. The composition of claim 1 wherein the first emulsifier and the second emulsifier, in combination, and anhydrous base are in a ratio ranging of about 12.333:1.

8. The composition of claim 1 wherein the first emulsifier is polyoxyethylene sorbitan monooleate.

9. The composition of claim 1 wherein the second emulsifier is tocopheryl polyethylene glycol succinate.

10. The composition of claim 1 wherein the anhydrous base is selected, either singly or in combination from the group comprising animal, vegetable, marine-based, and algae oils containing omega fatty acid.

11. The composition of claim 1 wherein the first emulsifier and the second emulsifier have individual HLB values in the range of about 10 to about 30.

12. The composition of claim 1 wherein the first emulsifier and the second emulsifier have individual HLB values in the range of about 12 to about 18.

13. The composition of claim 1 wherein the first emulsifier and second emulsifier, when in combination, have an HLB value in the range of about 14 to about 14.6.

14. The composition of claim 1 further including a solubilizing agent.

15. The solubilizing agent of claim 14 selected from the group, either singly or in combination, comprising polyethoxylated castor oil, polyethylene glycols, propylene glycol, fatty acids and esters, ethoxylated fatty acids and esters, alcohols, and their derivatives.

16. A microemulsion composition for increasing the bioavailability of statin drugs in humans and animals comprising polyoxyethylene sorbitan monooleate and a second emulsifier in a ratio ranging from about 1:1 to about 4:1 and a lipid, wherein the polyoxyethylene sorbitan monooleate and the second emulsifier, in combination, is in a ratio to the lipid ranging from about 99:1 to about 9:1.

17. The composition of claim 16 wherein the polyoxyethylene sorbitan monooleate and the second emulsifier are in a ratio of about 2:1.

18. The composition of claim 16 wherein the polyoxyethylene sorbitan monooleate and the second emulsifier are in a ratio of about 12.333:1.

19. The composition of claim 16 wherein the lipid is selected, either singly or in combination from the group comprising animal, vegetable, marine-based, and algae oils containing omega fatty acid.

20. The composition of claim 16 wherein the polyoxyethylene sorbitan monooleate and the second emulsifier have individual HLB values in the range of about 10 to about 30.

21. The composition of claim 16 wherein the polyoxyethylene sorbitan monooleate and the second emulsifier have individual HLB values in the range of about 12 to about 18.

22. The composition of claim 16 wherein the polyoxyethylene sorbitan monooleate and second emulsifier, when in combination, have an HLB value in the range of about 14 to about 14.6.

23. The composition of claim 16 further including a drug solubilized in the polyoxyethylene sorbitan monooleate, second emulsifier, and lipid.

24. The drug of claim 23 wherein statin drug is atarvostatin.

25. The composition of claim 16 further including a solubilizing agent.

26. The solubilizing agent of claim 25 selected from the group, either singly or in combination, comprising polyethoxylated castor oil, polyethylene glycols, propylene glycol, fatty acids and esters, ethoxylated fatty acids and esters, alcohols, and their derivatives.

27. A microemulsion composition for increasing the bioavailability of statin drugs in humans and animals comprising a first emulsifier and tocopheryl polyethylene glycol succinate in a ratio ranging from about 1:1 to about 4:1 and an anhydrous base, wherein the first emulsifier and the tocopheryl polyethylene glycol succinate, in combination, is in a ratio to the anhydrous base ranging from about 99:1 to about 9:1.

28. The composition of claim 27 wherein the first emulsifier and the tocopheryl polyethylene glycol succinate are in a ratio of about 2:1.

29. The composition of claim 27 wherein the first emulsifier and the tocopheryl polyethylene glycol succinate are in a ratio of about 12.333:1.

30. The composition of claim 27 wherein the anhydrous base is selected, either singly or in combination from the group comprising animal, vegetable, marine-based, and algae oils containing omega fatty acid.

31. The composition of claim 27 wherein the first emulsifier and the tocopheryl polyethylene glycol succinate have individual HLB values in the range of about 10 to about 30.

32. The composition of claim 27 wherein the first emulsifier and the tocopheryl polyethylene glycol succinate have individual HLB values in the range of about 12 to about 18.

33. The compostion of claim 27 wherein the first emulsifier and tocopheryl polyethylene glycol succinate, when in combination, have an HLB value in the range of about 14 to about 14.6.

34. The composition of claim 27 further including a drug solubilized in the mixture of the first emulsifier, tocopheryl polyethylene glycol succinate, and anhydrous base.

35. The composition of claim 34 wherein the statin drug is atarvostatin.

36. The composition of claim 27 further including a solubilizing agent.

37. The solubilizing agent of claim 36 selected from the group, either singly or in combination, comprising polyethoxylated castor oil, polyethylene glycols, propylene glycol, fatty acids and esters, ethoxylated fatty acids and esters, alcohols, and derivatives.

38. A composition for increasing the bioavailability of statin drugs in humans and animals comprising a microemulsion further comprising a first emulsifier, a second emulsifier, a lipid containing omega fatty acids and a statin drug.

39. The composition of claim 38 wherein the first emulsifier and the second emulsifier are in a ratio ranging from about 1:1 to about 4:1.

40. The composition of claim 38 wherein the first emulsifier and the second emulsifier are in a ratio of about 2:1.

41. The composition of claim 38 wherein the first emulsifier and the second emulsifier, in combination, and lipid are in a ratio ranging from about 99:1 to about 9:1.

42. The composition of claim 38 wherein the first emulsifier and the second emulsifier, in combination, and lipid are in a ratio ranging of about 12.333:1.

43. The composition of claim 38 wherein the first emulsifier is polyoxyethylene sorbitan monooleate.

44. The composition of claim 38 wherein the second emulsifier is tocopheryl polyethylene glycol succinate.

45. The composition of claim 38 wherein the lipid is selected, either singly or in combination from the group comprising animal, vegetable, marine-based, and algae oils.

46. The composition of claim 38 wherein the statin drug is atarvostatin.

47. The composition of claim 38 wherein the first emulsifier and the second emulsifier have individual HLB values in the range of about 10 to about 30.

48. The composition of claim 38 wherein the first emulsifier and the second emulsifier have individual HLB values in the range of about 12 to about 18.

49. The composition of claim 38 wherein the first emulsifier and second emulsifier, when in combination, have an HLB value in the range of about 14 to about 14.6.

50. The composition of claim 38 further including a solubilizing agent.

51. The solubilizing agent of claim 50 selected from the group, either singly or in combination, comprising polyethoxylated castor oil, polyethylene glycols, propylene glycol, fatty acids and esters, ethoxylated fatty acids and esters, alcohols, and their derivatives.

52. A method for increasing the bioavailability of a statin drug comprising the steps of:
combining a first emulsifier, a second emulsifier and an anhydrous base, the first emulsifier and second emulsifier, in combination, being in a ratio ranging from about 1:1 to 4:1 with a resulting HLB range of about 10 to about 30, and the ratio of the first emulsifier and second emulsifier, in combination, and anhydrous base being from about 99:1 to about 9:1;
solubilizing a statin drug in the combination of first emulsifier, a second emulsifier and anhydrous base, thereby creating a microemulsion;
providing the microemulsion to a patient;
having the microemulsion come into contact with water;
dispersing the solubilized drug; and,
having the drug absorbed.

53. The method of claim 52 including the step of selecting the first emulsifier as polyoxyethylene sorbitan monooleate.

54. The method of claim 52 including the step of selecting the second emulsifier as tocopheryl polyethylene glycol succinate.

55. The method of claim 52 including the step of selecting the anhydrous base, either singly or in combination, from the group comprising animal, vegetable, marine-based, and algae oils containing omega fatty acid.

56. The further step of claim 52 further including the step of selecting the statin drug atarvostatin.

57. The method of claim 52 further including the further step of adding a solubilizing agent to further enhance the solubilization of a drug.

58. The further step of claim 57 including the further step of selecting the solubilizing agent from the group, either singly or in combination, comprising polyethoxylated castor oil, polyethylene glycols, propylene glycol, fatty acids and esters, ethoxylated fatty acids and esters, alcohols, and derivatives.
